(19) United States
(12) Patent Application Publication
(10) Pub. No.: US 2022/0306245 A1
(43) Pub. Date: Sep. 29, 2022

(54) BUOY FOR CHECKING CONDITION OF A PERSON OR AN OBJECT TIED TO THE SAID BUOY

(71) Applicant: Seavision d.o.o., Dobrova (SI)

(72) Inventors: Anze PEKLENK, Dobrova (SI); Andrej PODBEVSEK, Kamnik (SI); Matija JASAROV, Celje (SI); Matic TOMAZEVIC, Ig (SI)

(21) Appl. No.: 17/705,280

(22) Filed: Mar. 26, 2022

(30) Foreign Application Priority Data
Mar. 26, 2021 (SI) P-202100061

(51) Int. Cl. B63B 22/00; A61B 5/00; B63B 45/04; B63B 27/18; B63B 21/20; B63B 5/24; G01S 19/01; H04N 7/18; H04N 5/225; H02S 10/40

(52) U.S. Cl. CPC B63B 22/00; A61B 5/6887; B63B 45/04; B63B 27/18; B63B 21/20; B63B 5/24; G01S 19/01; H04N 7/183; H04N 5/2257; H02S 10/40; B63B 2021/203

(57) ABSTRACT

The present invention belongs to the nautical field, specifically to the area of buoys. The invention relates to a buoy for checking the condition of a person or an object tied to the said buoy. The essence of the buoy according to the invention is in that it comprises a casing, which floats on the water surface, and a capsule with a camera and lights, which can be separated from the casing with a UTP cable and can be lowered down to an anchor, an object or a person. The wire rope, which is connected to the lower part of the capsule can be connected to any anchor, a fixed object on the sea bed or a person. The camera captures images and/or video and sends data via the UTP cable to the processor, and the processors sends data via wireless network (WiFi or similar) to the mobile application installed on the user device, which is preferably a smart phone, a tablet, computer or a similar device. The user can visually check the condition of the anchor and simultaneously monitor the anchor position via one or more satellite systems for global navigation, and the position is displayed graphically and numerically on the application on a mobile telephone, a tablet or a computer.

### FIELD

The present invention belongs to the nautical field, specifically to the field of buoys for tying vessels. The invention relates to a buoy for checking the condition of a person or an object tied to the said buoy.

### BACKGROUND

A buoy is a floating structure used for different purposes. Several different types of buoys are available: a communication buoy, a navigation buoy, a sonar buoy, a markation buoy, a weather buoy and a diving buoy, wherein they can be anchored or free-floating. A buoy can be used for marking vessel quays, especially outside marina. All buoys have a common characteristic—they mark a certain location, and they can emit and receive data important for navigation, measurements or communication.

The buoy attached to a vessel mooring or in most cases to a vessel, which is anchored with an anchor, it is important to monitor the condition of the mooring as well as the condition of the vessel's anchor. While the state of the mooring (presence of vessel) can be monitored with the solution described in the European patent EP 2 531 981 B1, it does not enable monitoring of the anchor condition or any other events under the water surface. Some other solutions can only monitor the vessel location, which is not important for the underwater condition of anchor, since its micro location can change in such a manner that the anchor may become entangled or stuck. The technical problem, solved by the said invention, is construction of a buoy, which will enable checking the condition of person or object fastened (tied or attached) to the said buoy.

For this purpose underwater drones or cameras, such as described in the patent application US20050036031, may be used, however, they must be monitored by the user at all times otherwise important data is lost in important moments. The preparation of currently known underwater drones is time-consuming. Additionally, they need to locate the desired object or the person in the water in order to allow monitoring. When the drone is back on land or on vessel the monitoring of the person or the object is lost. The camera, as described in the patent application US20050036031 is intended mostly for observing sea life and is connected via cable to a specific device, which requires continuous monitoring. It does not display the object's or person's position and it is very time-consuming for preparation. When the water depth is changing, it is also important to monitor and unwind the cable in order to maintain same position. The present invention solves listed shortcomings and disadvantages.

The patent application US2015116496A1 describes an anchor, equipped with a camera, a sensor and a light. The system connected to the camera, sensor and light on the anchor guarantees anchor monitoring in real time and enables notifications regarding the anchor state.

Patent U.S. Pat. No. 9,729,253 discloses a system with an underwater housing, which comprises an underwater wireless communication module, connected to the camera and an above-water housing with an above-water wire communication module, wherein both communication modules communicate via a cable. Camera captures photographs or video, which is forwarded via modules to a mobile device to monitor the conditions underwater.

Patent application AU2014100760A describes a safety buoy for use in marines, which has an electronic control unit and a radio transmitter that launches communication with the satellite network and sends out a distress call when activated with the electronic unit.

Document AT517793 describes a device for optical control of water bodies, especially bodies under water, the said device being provided with an attached buoy, wherein the rope of the buoy has a waterproof housing with installed cameras directed in all directions, along with a compass. The device can also include a sensor for determining camera position. An image can be captured with a trigger button via a transponder and suitable electronics.

Patent application U.S. Pat. No. 5,803,780 discloses a buoy for marking, which can be used to simply mark a location at the sea bed. The buoy comprises a control mechanism, which releases a weight by using the optimal rope amount.

Patent application U.S. Pat. No. 7,034,680 describes a device for monitoring anchor or anchor rope, which includes a measuring device, which uses one or more sensors to determine a status of one or more points on the anchor or the anchor rope with regards to the vessel and generates a signal to trigger an alarm when it deviates from the pre-set values.

Patent application CA2088088 relates to a buoy for marking a location of an underwater object, especially a sunken vessel's anchor. A spherical buoy housing is connected via a nylon rope and an attachment to the anchor before it submerges. The length of the unwinding rope is controlled by a mechanism, so that the location of the buoy is not changed due to waves or other water movement, which enables reliable marking of the location of the anchor under water.

The present invention differs from the above solutions in its construction.

### SUMMARY

In a broad aspect of the invention, embodiments of the buoy comprises:
a casing with an upper part with solar cells for ensuring energy for function and a lower part;
a watertight and/or waterproof chamber for key functional components of the buoy, wherein said chamber is installed in the interior of the buoy casing and houses a battery, a processor, the positioning module for determining geographic position via one or more systems for global navigation (GNSS, e.g. GALILEO, GPS, GLONAS, BEIDOU), a radio WiFi transmitter and receiver and a module for data transfer via network of mobile communication (e.g. LTE, 5G);
a capsule with the camera and at least one light, preferably more lights; most preferably the lights are a combination of IR and LED lights, wherein the capsule is connected to the bottom part of the casing in a separable manner;
an UTP/Ethernet cable, which connects to the buoy capsule with the casing of the buoy, where the cable is connected to the processor, wherein the cable unwinds and coils with a rotating mechanism, comprising:
   a reel, installed on an aluminium shaft, which acts as a rotating rotation axis, and inside which the UTP cable is installed, wherein the aluminium shaft is installed in the casing with holders up to a sliding ring;
   the sliding ring, which consists of rotating and non-rotating rotating part, and is arranged to accept electrical circle from the battery to the capsule and a video signal from the camera to the processor, wherein the UTP cable is set up through an aluminium shaft to the rotating part of the slide ring and via sliders in the slide ring to the non-rotating part of the slide ring, installed in the chamber;
   a bushing or a slide bearing installed on the aluminium shaft for ensuring unlimited rotation of the said shaft, and
   a Retractable tension spring, for ensuring automatic unwinding and winding the UTP cable on the reel; and
a wire rope with one end attached to the bottom part of the capsule, the other end being free and preferably equipped with a ring for attaching an anchor or any other object or a person.

### BRIEF DESCRIPTION OF THE DRAWINGS

FIG. 1 illustrates the buoy according to a possible embodiment with the capsule in the starting position;

FIG. 2 illustrates the buoy according to a possible embodiment with a lowered capsule;

FIG. 3 illustrates the interior of the buoy casing

FIG. 4 illustrates a section of the rotating mechanism;

FIG. 5a illustrates a section of the rotating mechanism and the chamber housing the key components;

FIG. 5b illustrates a section of the rotating mechanism, the chamber housing the key components and the capsule;

FIG. 6 illustrates a use of the buoy for marking location and monitoring the diver;

FIG. 7 illustrates a use of the buoy for marking location and monitoring the sea bed condition; and

FIG. 8 illustrates a use of the buoy for marking location and monitoring the anchor status used to anchor the vessel.

### DESCRIPTION

The essence of the buoy according to the invention is in that it comprises a casing, which floats on the water surface, a capsule with camera and lights, which can be separated from the casing with a UTP cable and lowered down to an anchor, an object or a person. The wire rope, which is connected to the lower part of the capsule can be connected to any anchor, fixed object on the sea bed or it can be attached to a person (diver) with the wire rope. The camera captures images and/or video and sends data via UTP cable to the processor, and the processor emits data via wireless network (WiFi or similar) to the mobile application installed on the user device, which is preferably a smart phone, a tablet, computer or a similar device. The user can visually check the condition of the anchor and simultaneously monitor the anchor position via one or several satellite systems for global navigation (GNSS), e.g. GALILEO, GPS, GLONASS, BEIDOU, and the position is displayed graphically and numerically on an application on mobile telephone, tablet or computer. The buoy sends out images and video to the application, as well as telemetric data on battery condition, turning on and off the camera and lights, and sends out data to the server online or in a computer cloud, making the data, images and video available via any device connected to the internet.

The buoy according to the invention preferably comprises:
the casing with an upper part with solar cells for ensuring energy for function and a lower part;
a watertight and/or waterproof chamber for key functional components of the buoy, wherein said chamber is installed in the interior of the buoy casing and houses a battery, a processor, the positioning module for determining geographic position via one or more systems for global navigation (GNSS, e.g. GALILEO, GPS, GLONAS, BEIDOU), a radio WiFi transmitter and receiver and a module for data transfer via network of mobile communication (e.g. LTE, 5G);
a capsule with the camera and at least one light, preferably more lights; most preferably the lights are a combination of IR and LED lights, wherein the capsule is connected to the bottom part of the casing in a separable manner;
an UTP/Ethernet cable, which connects to the buoy capsule with the casing of the buoy, where the cable is connected to the processor, wherein the cable unwinds and coils with a rotating mechanism, comprising:
   a reel, installed on an aluminium shaft, which acts as a rotating rotation axis, and inside which the UTP cable is installed, wherein the aluminium shaft is installed in the casing with holders up to a sliding ring;
   the sliding ring, which consists of rotating and non-rotating rotating part, and is arranged to accept electrical circle from the battery to the capsule and a video signal from the camera to the processor, wherein the UTP cable is set up through an aluminium shaft to the rotating part of the slide ring and via sliders in the slide ring to the non-rotating part of the slide ring, installed in the chamber;
   a bushing or a slide bearing installed on the aluminium shaft for ensuring unlimited rotation of the said shaft, and
   a retractable tension spring, for ensuring automatic unwinding and winding the UTP cable on the reel;
a wire rope with one end attached to the bottom part of the capsule, the other end being free and preferably equipped with a ring for attaching an anchor or any other object or a person.

The buoy casing is primarily made from ecologically processed, strong and resistant plastic mass, and both parts are assembled with threads and seals. The buoy is designed to preserve buoyancy even in case it submerges, because the watertight chamber for the key components in the buoy interior is an airtight sealed space and the air inside the buoy maintains it above water.

The upper part of the buoy can be partially transparent and can additionally comprise signal lights in case of danger or anchor movement. The upper part may also comprise solar cells for charging the battery, which is installed in the waterproof chamber for key components, wherein the solar cells can be installed on the casing, but are preferably provided in the interior under the transparent material of the upper part of the casing.

The lower part of the buoy has an opening at the bottom for the UTP/Ethernet cable. The bottom part of the buoy casing can have small holes at the bottom part, which enable draining of excess water, which remains in the buoy when lifted. The bottom part of the casing is designed to have a movable capsule with the camera and lights, or the casing is in its bottom part designed as a cavity, which can partially hide the capsule with the camera and lights.

The capsule can be separated from the buoy casing by unwinding the cable using the rotating mechanism, which operates in such a manner that upon attaching a load on the wire rope the capsule is lowered due to gravity, while in case the load is removed from the wire rope the UTP cable is automatically wound on the reel due to the tensile spring, which is attached at the end of aluminium shaft in a fixed manner. The latter is attached to the two holders and together with the bushing enables the reel to be stable and attached in the interior of the buoy casing, wherein the bushing enable shaft rotation of the shaft without traction. The sliding bearing is made from highly resistant plastics and ensures mechanical stability with lowest coefficients of traction without additional greasing and is resistant to sea water. One end of the aluminium shaft is attached to the sliding bearing, and the other part is installed to the spring with constant force in a fixed manner. The spring is made from a coiled steel wire, and its coils enable stretching and automatic contraction. In case the load from the wire rope is removed, the UTP cable is automatically wound back on the reel. In case the cable retracts, i.e. it is pulled out of the buoy casing, the pulling force must be higher than the force of the spring. This causes that the UTP cable is constantly tensed and that the buoy above the anchor is always in the same position above it. Even in the case of waves the spring is loosened and automatically winds the cable back to the reel. The holders of the aluminium shaft are attached with screws to the bottom part of the buoy casing, said holders being made from stainless steel.

The watertight and/or waterproof chamber for key components for buoy operation is installed in the interior of the buoy casing, preferably the chamber of key components is made from two parts and the bottom part is with screws attached to the bottom part of the buoy casing, and the cover is tightly attached with screws and seals to the bottom part of the chamber of key components, which are the battery, the processor, the positioning module for determining geographic position with one or more systems GNSS, radio WiFi transmitter and receiver and a module for data transfer vial networks of mobile communication (LTE, 5G). On the inner part at the side of the chamber cover an opening is provided, through which the non-rotating part of the slide ring is installed, said non-rotating part leading the UTP cable from the processor via the rotating mechanism to the capsule.

The watertight capsule is made from two parts, i.e. the upper part and bottom part of the capsule, which are connected to each other with screws and a seal. The bottom part, from the internal side, has a space for the camera and lights, preferably which are preferably IR and LED lights. The bottom part has a suitably designed opening and sealed transparent part so that the camera can take images, while the lights emit light through the transparent material. The entire capsule is covered in rubber (except the opening for camera and lights), which absorbs hits and protects the plastic part of the capsule mould from external influences. The external part of the bottom part of the capsule has an attachment element for the wire rope, which can be approximately 2 m long, and its free end is provided with a ring or any other suitable element to which an anchor or any other object or a person in the water can be attached or tied.

The camera in the capsule has the UTP cable that runs from the buoy casing, which fuels the camera with PoE (Power over Ethernet) electric current, which is used for sending image and video signal from the camera to the processor, which can send the data via WiFi to the application and via the module for data transfer via mobile network on the server. The camera is controlled via the cable with the processor and the application. The camera records in high resolution, and has an option of recording and saving individual photographs. All videos and images can be archived in the processor, the application and on a server. The camera enables visual control/monitoring of a certain object or a person underwater in real time. Preferably the camera has three modes of function. If the LED lights are on, the camera functions as usually, and if only IR lights are on, it operates in a night mode, wherein the camera can operate even with lights off if the visibility is suitable and enough natural light is available. The lights are charged with the UTP cable, and their function is controlled with an application on mobile devices or remotely via the server. Preferably, the application allows to choose turning on or off lights, which is communicated to the processor via a wireless connection, which forwards the information via the UTP cable to the camera in order to follow the processor commands. The camera is suitably connected to the lights so that it can adjust their function or light.

The application performs the following steps:
displaying video or image view of the anchor,
displaying anchor location on a map,
enabling adjustment of light and camera status on the buoy as described above,
enabling geofencing of the anchor up to 1 to 20 metres, which means that if the anchor deviates from the pre-set limits, the alarm is triggered thus notifying the user the anchor has moved outside the certain area, wherein the alarm can be auditory, image or in any other suitable alarm, and
archiving the image and video content by storing data on a suitable server.

For the preferred use of the buoy in monitoring the anchor condition and checking the anchoring at the same location, the application is used to set a geolocation, defined with a circle from 1 to 20 metres. In the moment the position is set it is archived by the positioning module, and fencing is implemented by a programme equipment with the processor. The drawn circle of the limitation is displayed on the map, and any potential additional movement of the buoy is monitored by the positioning module after setting up the position. If the anchor leaves the pre-set circle limit the alarm s triggered, and the processor acts accordingly. The buoy efficiently monitors the anchor movement on its own, its movement and together with capturing images and/or video enables safer anchoring of any vessel in any water, preferably in the sea, where movements, waves and tide are important factors for movement of vessels and anchors.

The process of using the buoy according to the invention comprises the following steps:

a) registration into the application with a username and a password,

b) connecting with the buoy or a specific buoy, if there are more, and naming the buoy;

c) displaying location of the selected buoy on a map;

d) connecting an anchor or an object tied to the buoy and lowering the anchor or the object into water, which causes automatic lowering or unwinding of the cable from the buoy;

e) once the anchor or the object is d on sea bed or at a certain depth, the unwinding stops or it winds back and lowers further via the self-winding system if needed, depending on the waves or movement of the anchor or the object;

f) if needed activation of lights and capturing image or video of object, connected to the buoy;

g) displaying image or video, wherein the lights can be controlled or adjusted (IR, LED and intensity) as described above;

h) if desired or optionally, allowing magnification, storing the images to a smartphone and access to gallery;

i) optional setting of geographic limits (geofencing) from 1 to 20 metres, which generates a circle around the buoy position;

j) possible triggering of the alarm if anchor moves out of the set circle;

k) if desired, turning off a camera and/or lights;

l) if necessary, lifting the anchor or object from the sea bed towards sea surface, which causes automatic winding of the UTP cable using the self-winding system; and

m) lifting the buoy out of the water.

The buoy according to the invention can be used to monitor underwater activity, especially on sea, lake or river bed. It is primarily intended for observing and monitoring the condition of the anchor, which is tied to the buoy, ensuring safe anchoring of the vessel anywhere in the sea, lake or river. Simultaneously, the buoy can be used to observe underwater life, a visual or image depiction of sea, lake or river bed. Nonetheless, the buoy according to the invention improves the safety of divers, since it can constantly monitor their dive and/or work and in case of problems ensures quick reactions.

The buoy for checking the condition of person or object tied to the said buoy according to the invention will be described in further detail based one exemplary embodiments and figures, which show:

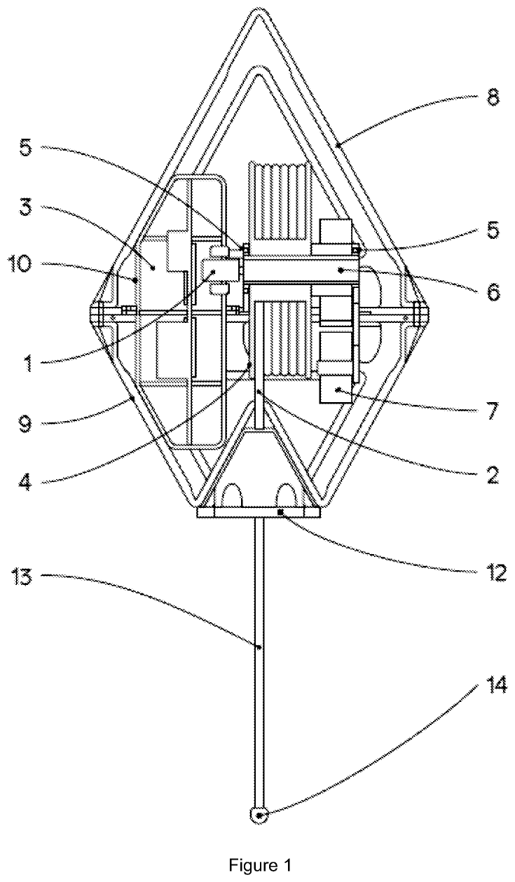

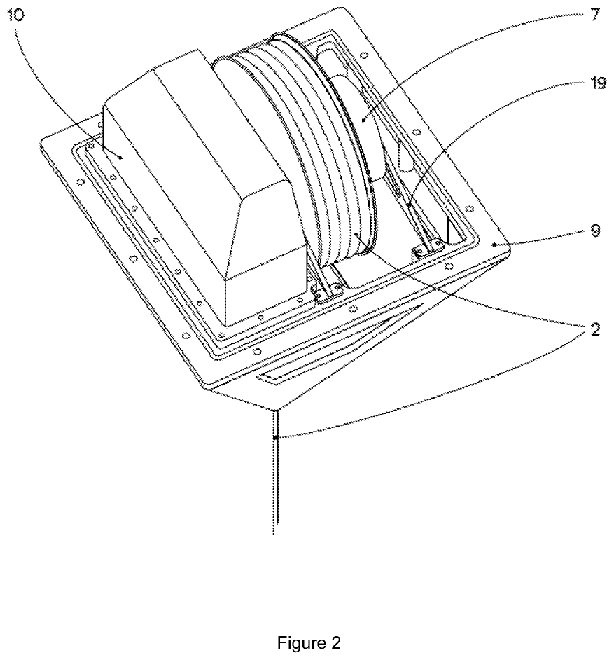

A possible embodiment of the buoy is shown FIGS. 1 and 2 and comprises:
a casing, which has an upper part 8 with solar cells 18 for guaranteeing energy for function and bottom part 9, wherein the casing in the upper part is made from see-through plastic material and the solar cells 18 installed on the inner side of the see-through upper part 8 of the buoy casing;
a watertight and/or waterproof chamber 10 for key components 3 for buoy function, which is installed in the casing interior 8, 9 of the buoy and which has installed battery, processor, positioning module, WiFi transmitter and LTE module, wherein the positioning module is any GNSS.
capsules 12 with a camera 15 and lights 16, 17, primarily the lights are a combination of IR 16 and led lights 17, wherein the capsule 12 is separable connected to the bottom part of the casing 9;
an UTP/ethernet cable 2, which connects the capsule 12 to the buoy casing 8, 9 which is connected to the processor, wherein the cable 2 unwinds and coils with the help of a rotating mechanism, comprising:
   a reel, installed to the aluminium shaft 6, which acts as a rotating axis, and has installed a UTP cable 2, wherein the aluminium shaft 6 is installed in the casing with the help of the pylons 19 up to the slide ring 1;
   a slide ring 1, which consists of rotating and non-rotating part, and it is adjusted to accept electrical circle from the battery to the capsule 12 and a video signal from the camera to the processor, wherein the UTP cable 2 is set up through an aluminium shaft 6 to the rotating part of the slide ring 1 and via sliders in the slide ring to the non-rotating part of the slide ring, installed in the chamber 10;
   a bushing 5 or slide bearing installed to the aluminium pipe 6 for guaranteeing undisturbed rotating of the said pipe 6, and
   a retractable tensile spring 7, which enables automatic reeling and winding the UTP cable 2 on the reel;
a wire rope 13 attached to the bottom part of the capsule 12, and the free part of the wire rope is primarily equipped with a hoop 14 for attaching an anchor or any other object or person.

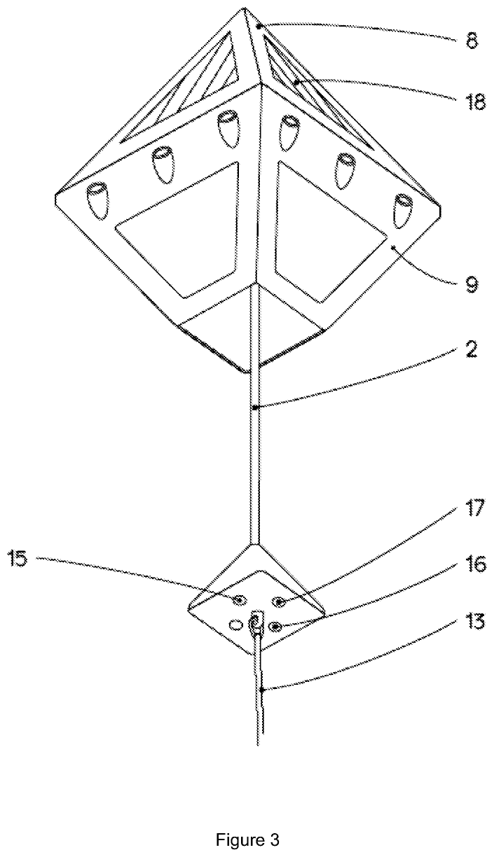

The buoy casing 8, 9 is made from ecologically processed, strong and resistant plastic mass, and both parts are assembled with threads and seals. The upper part 8 also comprises solar cells 18 for charging the battery, which is installed in the waterproof chamber 10 for key components 3, wherein the solar cells 18 are installed below the transparent material of the upper part 8 of the housing. The bottom part 9 of the buoy casing has an opening at the bottom, through which the UTP/Ethernet cable 2 runs, and at the same time the bottom part has a designed cavity to hide the capsule 12 with the camera 13 and lights 16, 17. The capsule 12 can be lowered as shown in FIG. 3, wherein it remains connected to the buoy with the UTP cable 2.

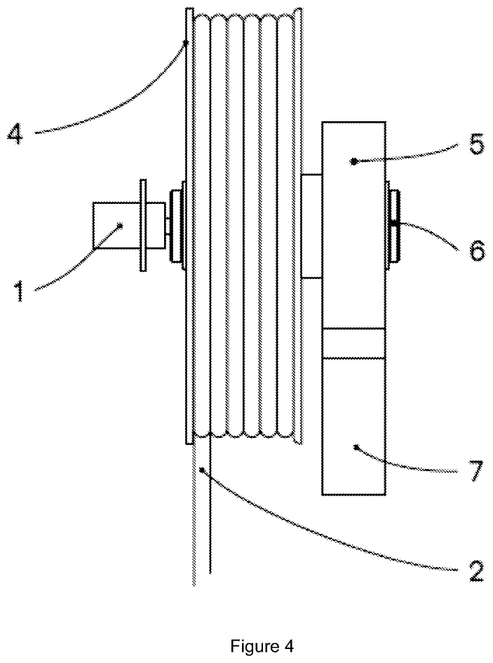

FIG. 4 displays the section of rotating mechanism, comprising:
the reel, installed on an aluminium shaft 6, inside which the UTP cable 2 installed,
the slide ring 1, which comprises a rotating and a non-rotating part, wherein the latter is installed in the chamber 10,
the bushing 5 or a slide bearing installed on the aluminium shaft 6 for ensuring unlimited rotation of the said shaft, and
the self-winding tension spring 7.

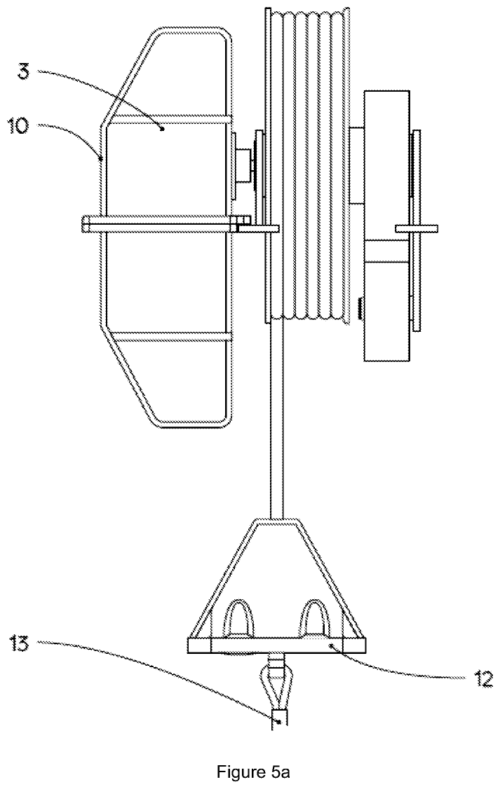

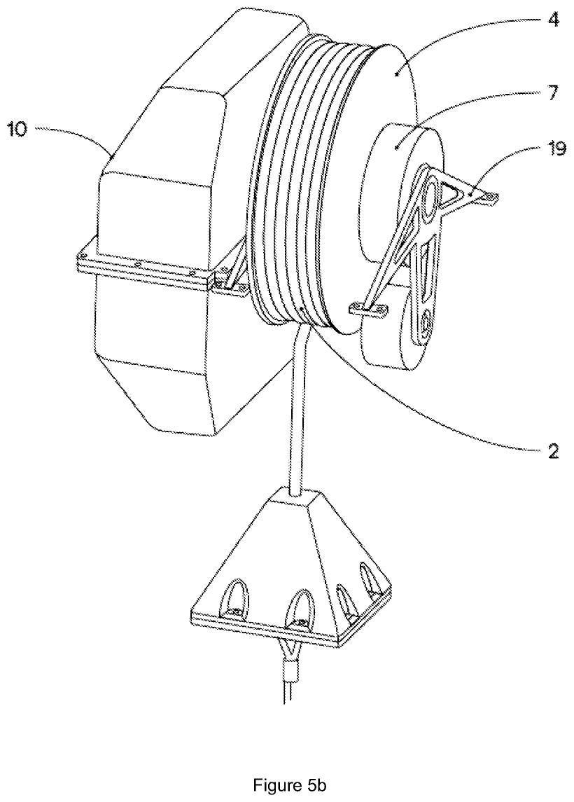

FIGS. 5a and 5b in addition to the rotating mechanism also the capsule 12 and the chamber 10 are shown, while the non-rotating part of the slide ring is not visible.

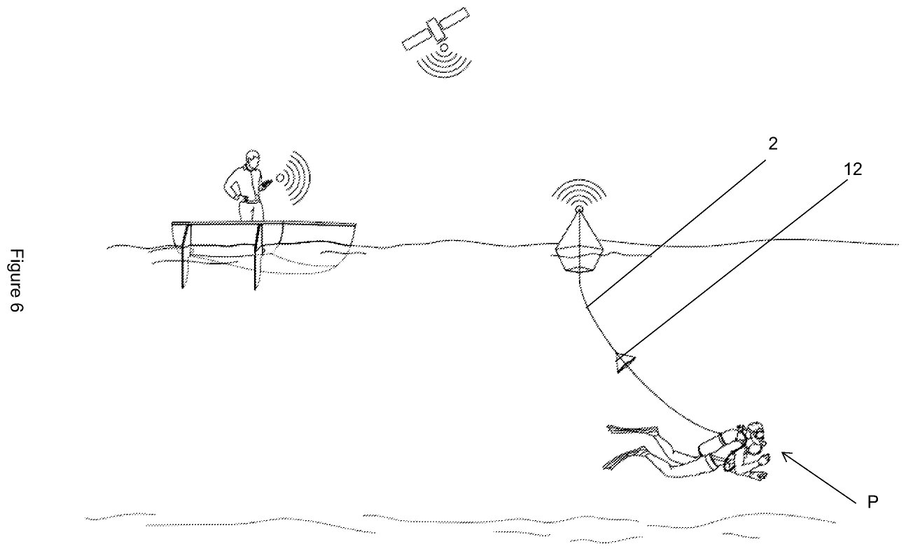

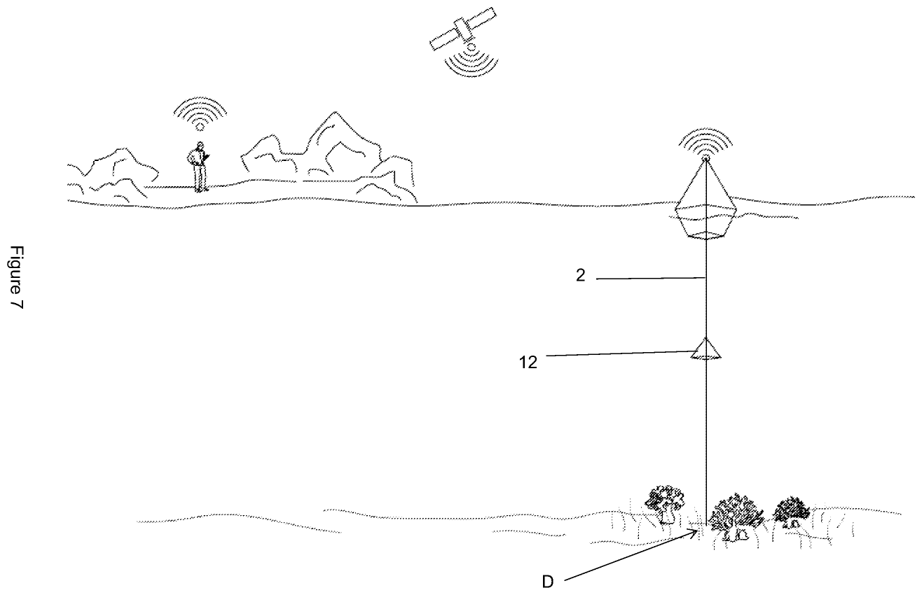

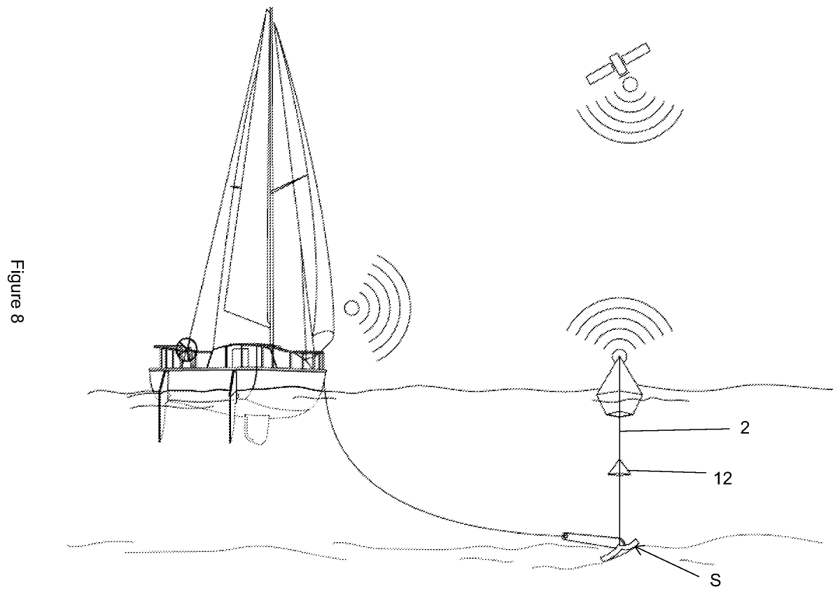

FIGS. 6 to 8 show some possible ways of use, e.g. use of the buoy for marking location and monitoring the condition of a diver P, a location at sea bed D or an anchor S, which is used to anchor a vessel. In all cases the wire rope of the capsule 12 is tied or attached to the diver P, and the anchor S or wire rope is attached to the object at the sea bed, which causes separation on the capsule 12 from rest of the buoy, i.e. the capsule 12 is located lower than the buoy floating on the water surface. The UTP cable 2, which connects the buoy with the lowered capsule 12 enables transfer of energy for operation of the camera and lights as well as data transfer of capturing images or video with camera, and also for controlling the components of the capsule 12.

1. Buoy for checking condition of a person or an object tied to the said buoy, characterised in that the buoy comprises:
a casing (8, 9), which is arranged to float on sea surface, and
a capsule (12) with a camera (15) and lights (16, 17), which can be separated from the casing (8, 9) with a UTP cable (2) and is arranged to be lowered towards an anchor, an object or a person, wherein the cable (2) unwinds and winds using a rotating mechanism, which comprises a sliding ring (1) comprising a rotating and non-rotating part which enable unwinding of the UTP cable (2), which ensures smooth transfer of electric energy and video signal via UTP cable (2) from a camera (15) to a watertight and/or waterproof chamber (10) for housing key components (3) and in vice-versa,
wherein the camera (15) is arranged to capture images and/or video and send data via the UTP cable (2) to a processor, said processor being arranged to send data via wireless network (WiFi or similar), to a mobile application installed on a user device, which is preferably a smart phone, a tablet, computer or a similar device.

2. The buoy according to claim 1, characterised in that it comprises:
the casing (8, 9) with an upper part (8) with solar cells (18) for ensuring energy for function and a lower part (9);
the watertight and/or waterproof chamber (10) for key components (3) for operation of the buoy, wherein said chamber (10) is installed in the interior of the buoy casing and inside which the battery, the processor, a positioning module for determining geographic position via one or more systems for global navigation (GNSS, e.g. GALILEO, GPS, GLONAS, BEIDOU), radio WiFi transmitter and a receiver and a module for data transfer via network of mobile communication (e.g. LTE, 5G) are installed,
the capsule (12) with the camera (15) and at least one light (16, 17), preferably more lights; preferably the lights are a combination of IR (16) and LED lights (17), wherein the capsule (12) is connected to the bottom part (9) of the casing in a separable manner;
the UTP/Ethernet cable (2), which connects the capsule (12) to the buoy casing, which is connected to the processor, wherein the cable (2) unwinds and coils with the help of a rotating mechanism, comprising:
a reel, installed to the aluminium shaft (6), which acts as a rotating axis, and has installed a UTP cable (2), wherein the aluminium shaft (6) is installed in the casing with the help of the pylons (19) up to a slide ring (1);
the slide ring (1), which consists of rotating and non-rotating part, and it is adjusted to accept electrical circle from the battery to the capsule (12) and a video signal from the camera (15) to the processor, wherein the UTP cable (2) is set up through an aluminium shaft (6) to the rotating part of the slide ring (1) and via sliders in the slide ring (1) to the non-rotating part of the slide ring, installed in the chamber (10);
Bushing (5) or slide bearing installed to the aluminium pipe (6) for guaranteeing undisturbed rotating of the said pipe, and
Retractable tensile springs (7), which enables automatic reeling and winding the UTP cable (2) on the reel;
Wire rope (13) attached to the bottom part of the capsule (12), and the free part of the wire rope (13) is primarily equipped with a hoop for attaching an anchor (S) or any other object (D) or person (P).

3. The buoy according to claim 2, characterised in that the positioning module is one or several systems for global navigation (GNSS) selected in a group consisting of GALILEO, GPS, GLONAS, BEIDOU and any combination thereof.

4. The buoy according to claim 1, characterised in that the casing (8, 9) of the buoy is made from ecologically processed, strong and resistant plastic mass, and that both parts (8,9) of the casing are assembled with threads and seals.

5. The buoy according to claim 2, characterised in that the upper part (8) of the buoy is partially transparent and can additionally include signal lights in case of danger or movement of anchor, and in that solar cells (18) can be installed on the casing, preferably the solar cells (18) are installed in the interior of the casing underneath a transparent material of the upper part (8) of the casing.

6. The buoy according to claim 2, characterised in that the bottom part (9) of the buoy casing has an opening at the bottom, through which the UTP/Ethernet cable (2) runs, and the bottom part (9) is designed to allow installation of the moveable capsule (12) with the camera (15) and lights (16, 17); or the casing (9) is in the bottom part designed as a cavity, which can at least partially enclose the capsule (12) with the camera (15) and lights (16, 17).

7. The buoy according to claim 1, characterised in that the capsule (12) can be separated from the buoy casing (8, 9) by unwinding the cable (2) with the rotating mechanism, which acts by attaching to the weight on the wire rope (13) and due to gravity the capsule (12) moves downwards by unwinding cable (2), while when weight is removed on the wire rope (13) the cable (2) is automatically winds on the reel due to function of the tensile spring (7), which is fixed attached at the end of aluminium shaft (6), attached to the two pylons (19) and along with the bushing (5) enables the reel to be stable and attached in the interior of the buoy casing, and the bushing (6) enables shaft rotating without traction, and end of the aluminium shaft is attached to the slide bearing, and the other part is fixed installed to the retractable spring (7) with constant force that is made from coiled steel ribbon, and the coils of the spring enable stretching and automatic contraction.

8. The buoy according to claim 1, characterised in that the watertight and/or waterproof chamber (10) for key components for buoy function is installed in the interior of the buoy casing, and predominantly the chamber (10) of key components is in two parts and the bottom part (11) with screws is reeled in the bottom part (9) of the buoy casing, and the cover is tightly attached with screws and seals to the bottom part of the chamber of key components, such as battery, processor, positioning module for determining geographic position via one or more systems GALILEO, GPS, and/or GLONAS, radio WiFi transmitter and receiver and a module for data transfer vial networks of mobile communication (LTE, 5G), and the inner part by the side of the chamber cover has an opening, which has a non-rotating part of the slide ring (1), which leads the UTP cable (2) from the processor via the rotating mechanism to the capsule (12).

9. The buoy according to claim 1, characterised in that the watertight capsule (12) is made from the upper part and bottom part of the capsule (12), which are assembled with screws and a seal, wherein the bottom part has a suitably designed opening and a sealed transparent part so that the camera (15) can capture images, while the lights (16, 17) emit light through the transparent material.

10. The buoy according to claim 1, characterised in that the entire capsule (12), except for the opening for the camera (15) and lights (16, 17), is covered in rubber, which absorbs hits and protects the plastic part of the capsule (12) mould against external influences.

11. The buoy according to claim 1, characterised in that it is arranged to use the processor and the application to control the camera (15) via the UTP cable (2), said camera (15) enabling visual control/monitoring of a certain object (S, D) or a person (P) underwater in real time, wherein all recordings and images can be archived in the processor, the application and on a server.

12. The buoy according to claim 1, characterised in that the camera has three modes of function:
If the LED lights (17) are on, the camera (15) functions normally,
if only IR lights (16) are on, the camera (15) operates in a night mode,
the camera (15) operates even when the lights are off, if visibility is suitable and enough natural light is available.

13. The buoy according to claim 1, characterised in that the application performs at least the following steps:
Displays video or image view of the anchor (S),
Displays anchor (S) location on the map,
Enables adjusting conditions of light (16, 17) and camera (15) on the buoy as described above,
Enables the geofencing of the anchor (S) up to 1 to 20 metres, which means if the anchor (S) deviates from the pre-set limits, the alarm goes off, notifying the user the anchor has moved outside the certain area, wherein the alarm can be auditory, image or in any way suitable and
It archives the image and video content by storing to the suitable server.

14. The buoy according to claim 13, characterised in that the application is arranged in such a manner that for preferred use of the buoy in monitoring the anchor (S) condition and checking the anchoring (S) at the same location, the application is used to set a geolocation, thus defining a circle with a radius from 1 to 20 metres, wherein in the moment the position is set, is the position is archived by the positioning module, and fencing is implemented by a software with the processor thus drawing a limitation circle on the map, wherein any potential additional movement of the buoy is monitored by the positioning module after setting up the position, and wherein the anchor oversteps the pre-set circle limit the alarm is arranged to be triggered, wherein the processor is arranged to act accordingly.

15. The process of using the buoy according to claim 1, said process comprising the following steps:
a) Registration into an application with a username and password;
b) Connecting with the buoy or a specific buoy, if there are more, and naming the buoy;
c) Displaying location of buoy on map;
d) Connecting an anchor (S) or object (D) or person (P) tied to the buoy and descend into water, which causes automatic descending or unwinding of cable from buoy;
e) Once the anchor (S) or object (D) or person (P) is placed on sea bed or at certain depth the unwinding stops or it coils back and lowers further via self-coiling system if needed, depending on the waves or movement of anchor or object;
f) Optionally, activation of light and capturing image or video of object, connected to buoy;
g) Displaying image or video with the option of managing and adjusting lights (IR, LED and intensity) as described above;
h) optionally, the option of enhancing and archiving images to the smartphone and access to gallery;
i) Optional setting of geographic limits (geofencing) from 1 to 20 metres, drawing a circle around the buoy position;
j) Possible alarm triggering when anchor moves out of the set circle;
k) optionally, turning off a camera (15) and/or lights (16, 17);
l) optionally, lifting the anchor or object from the sea bed towards sea surface, which is managed by automatically coiling the UTP cable (2) with the assistance of self-coiling system (7); and
m) lifting the buoy from the water.
